(19) United States
(12) Patent Application Publication
(10) Pub. No.: US 2007/0154526 A1
(43) Pub. Date: Jul. 5, 2007

(54) Ingestible carrier for medications and structure and method for making

(76) Inventor: Charles P. Hickey, Monroe, WI (US)

Correspondence Address: BOYLE FREDRICKSON NEWHOLM STEIN & GRATZ, S.C., 250 E. WISCONSIN AVENUE, SUITE 1030, MILWAUKEE, WI, 53202, US

(21) Appl. No.: 11/324,653

(22) Filed: Jan. 3, 2006

(51) Int. Cl. A61K 47/00

(52) U.S. Cl. 424/439; 425/450.1

(57) ABSTRACT

The present invention is an ingestible carrier for a medication to be taken orally by an individual. The carrier includes a cavity into which one or more medications can be inserted and frictionally retained by the carrier. The carrier is formed of an easily ingestible material and is shaped in a suitable mold to include the cavity within which the medication can be positioned and retained. The carrier can be easily formed in a mold to enable the dosage form to be quickly easily made available for use.

### FIELD OF THE INVENTION

The present invention relates to oral medications, and more particularly to an ingestible carrier for medication and a method for forming the carrier for the medications that allows the medications to more easily be ingested.

### BACKGROUND OF THE INVENTION

With the developments in the field of pharmaceuticals, there are an increasing number of medications available for oral administration for a wide variety of conditions. These medications come in various forms that can be ingested orally, with the majority being of a size capable of being relatively easily swallowed by an individual.

However, due to the necessary dosage amounts for certain medications, or the particular form of the medication, the medications often times are not able to be readily swallowed by an individual due to the overlarge size of these medications. In addition, certain individuals are not capable, for various reasons, of easily swallowing medications that are required for the health of the individual.

In order to increase the ease of orally administering medications to individuals, a number of different dosage forms of the medications have been developed. The majority of these dosage forms include various coatings on the exterior of the medications, which enable the medications to be more easily swallowed by an individual. Nevertheless, on many occasions, these improved dosage forms of the medications still present certain difficulties to people taking the oral medication dosages.

Therefore, it is desirable to develop a carrier to assist in the ingestion of various oral medications and a structure and method of forming the carrier, which allows for oral medications to be taken in a quick and easy manner by individuals previously unable to do so.

### SUMMARY OF THE INVENTION

According to a primary aspect of the present invention, an ingestible carrier is provided which is created by a moldable and ingestible material that includes a recess formed within the material and in which the medication can be positioned. The recess enables the medication dose or dosages to be positioned completely within the carrier such that the medication can easily be ingested along with the carrier. The recess formed within the carrier is dimensioned to enable the medication or medications to be securely held within the recess, such that the medication cannot easily become dislodged from within the recess once positioned therein. This ensures that the medication is ingested along with the carrier without becoming separated from the carrier.

According to another aspect of the present invention, a mold structure is provided which allows for the formation of the carrier complete with the recess within the carrier in a simple manner. The mold can be configured to form any number of carriers at a single time, such that a number of different medications can be taken by different individuals at the same time. The mold has a very simple construction that allows it to be easily utilized to form the carrier by placing the ingestible material into the mold, and can be easily cleaned after use for additional uses to form ingestible carriers for oral medications.

Numerous other aspects, features and advantages of the present invention will be made apparent from the following detailed description taken together with the drawing figures.

### BRIEF DESCRIPTION OF THE DRAWINGS

The drawings illustrate the best mode currently contemplated as practicing the present invention.

In the drawings:

FIG. 1 is an isometric view of an adjustable carrier constructed according to the present invention;

FIG. 2 is an isometric view of a mold utilized to construct the adjustable carrier of FIG. 1;

FIG. 3 is a cross-sectional view along line 3-3 of FIG. 2;

FIG. 4 is a cross-sectional view along line 4-4 of FIG. 2;

FIG. 5 is a cross-sectional view illustrating the insertion of the medication into the adjustable carrier of FIG. 1; and

FIG. 6 is an isometric view of the molds utilized to form the carrier of FIG. 1 in a stacked configuration.

### DETAILED DESCRIPTION OF THE INVENTION

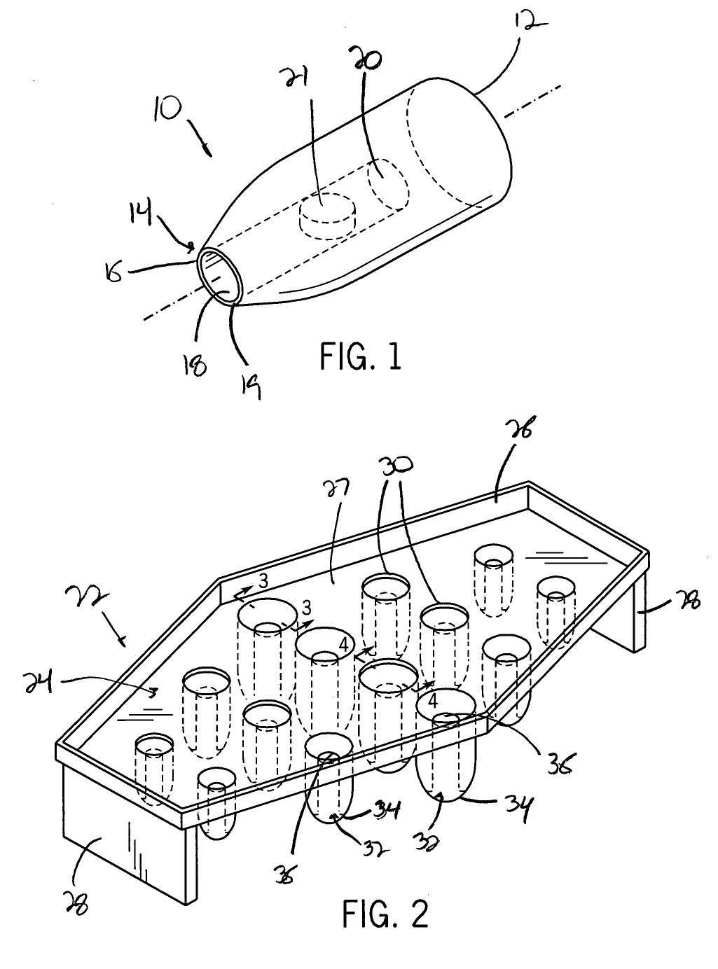

With reference now to the drawing figures in which like reference numerals designate like parts throughout the disclosure, an ingestible carrier constructed according to the present invention is illustrated generally at 10 in FIG. 1. The carrier 10 can have any suitable and easily ingestible shape, but the preferred embodiment is bullet-shaped and includes a flat side 12 and a tapered side 14. The tapered side 14 defines a narrow end 16 opposite the flat side 12 that radially expands in a gradual manner as the tapered side 14 extends towards the flat side 12.

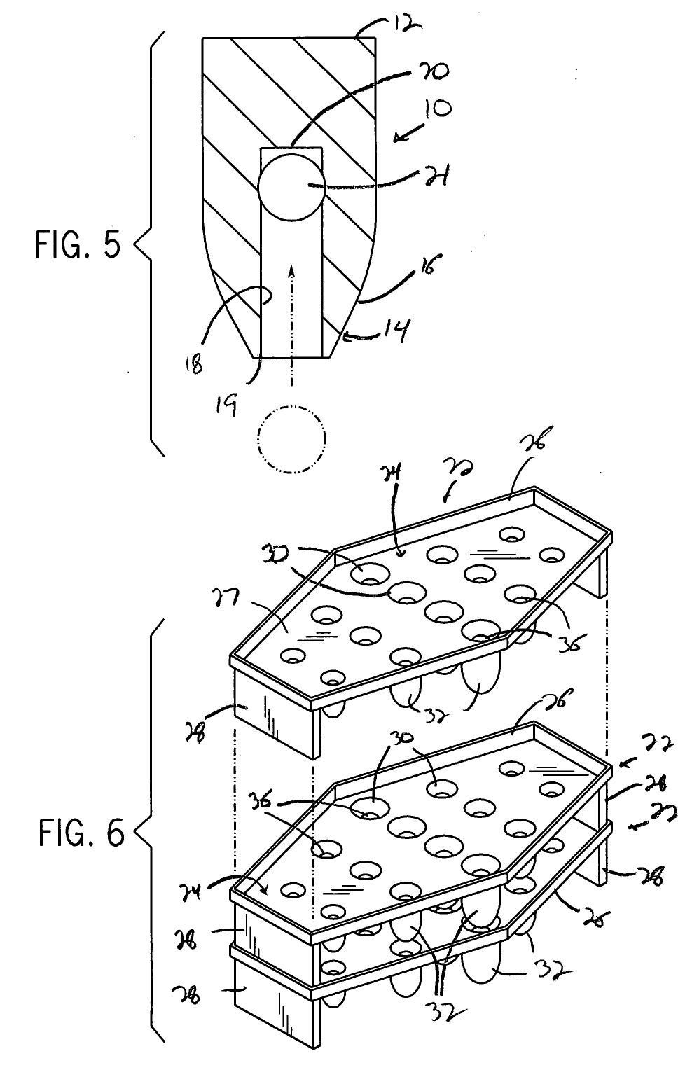

The narrow end 16 defines an interior recess or cavity 18 within which medication 21, such as a liquid medication, pill, tablet or capsule, can be positioned. The size of the cavity 18 is preferably slightly smaller in diameter than a conventional pill, table or capsule. As a result, when a medication in form of a pill, tablet or capsule is positioned within the cavity 18, the medication 21 is engaged by the sides of the cavity 18 such that the medication 21 is frictionally held within the cavity 18 once positioned therein, as best shown in FIG. 5. Preferably, the cavity 18 is cylindrical in shape, such that a wide variety of conventional medications can be positioned therein. Also, the cavity 18 defines an open end 19 at the tapered side 14, and a closed end 20 disposed within the carrier 10. The distance between the closed end 20 and the open end 19 is preferably sufficient to accommodate at least a pair of tablets or capsules 21 within the cavity 18. However, the cavity 18 can be shortened or lengthened to accommodate varying numbers of medication forms therein. Also, additional cavities 18 can be formed in other portions of the carrier 10 to further enable the carrier 10 to accommodate additional medications 21. The cavity 18 can also be adapted to retain liquid medications, such as by including an amount of an absorbent material (not shown) within the cavity 18 capable of retaining a liquid medication therein, and preventing the medication from flowing out of the cavity 18. The carrier 10 can also include a suitable plug (not shown) that is positionable within the open end 19 of the cavity 18 once the liquid medication is positioned therein to maintain the liquid medication within the cavity 18. The plug can be formed of the same material as the carrier 10, or another ingestible material, or by another medication 21 engaged within the cavity 18 over the liquid medication.

Additionally, the overall size of the carrier 10 can be selected to be any easily ingestible size that also can define a cavity 18 therein that is capable of receiving the medication 21 either entirely or at least partially therein. Preferably, the size of the overall carrier 10 is selected based on the size or age of the individual who will be ingesting the carrier 10, while the size of the cavity 18 remains the same diameter to accommodate and frictionally engage the medication 21.

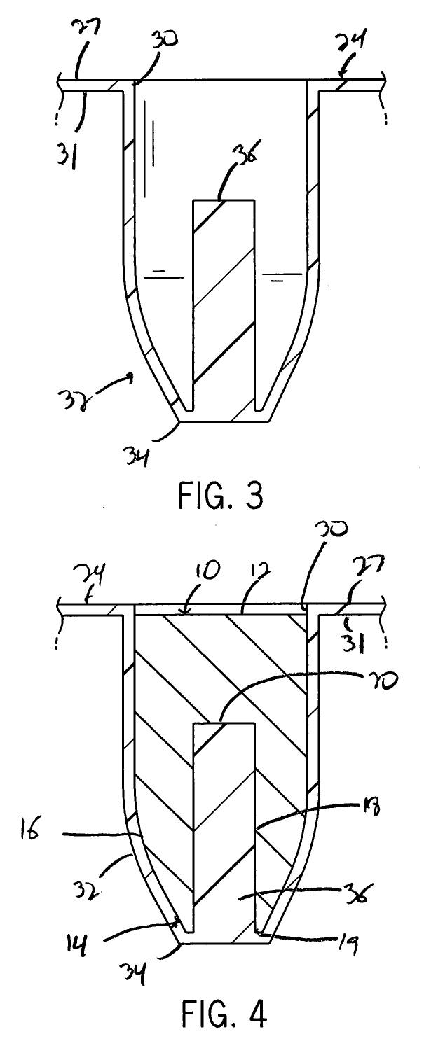

Referring now to FIGS. 2-4, a mold 22 utilized in the formation of the carrier 10 is illustrated. The mold 22 includes a central panel 24 surrounded by a peripheral rim 26 that extends around a top surface 27 of the panel 24 to provide a tray-like structure for the mold 22. The central panel 24 is supported at opposite ends by a pair of legs 28 extending downwardly from the central panel 24. The central panel 24, peripheral rim 26 and legs 28 forming the mold 22 are preferably formed of a lightweight, durable material, such as a plastic material. Further, the material used to form the mold 22 must be capable of withstanding the required amount of temperature variation required to form the carrier 10 within the mold 22. Also, when the central panel 24, rim 26 and legs 28 are formed of a plastic material, the components of the mold 22 can be integrally formed, or separately from one another, for later connection to one another in any suitable manner, such as by using an adhesive or by sonic welding, among others, to form the mold 22.

The panel 24 also includes a number of apertures 30 spaced about the panel 24. Each aperture 30 is surrounded on the lower surface 31 of the panel 24 by a downwardly extending form 32 in the desired shape of the carrier 10. At the lower end 34 of the form 32 opposite the lower surface 31 of the panel 24, the form 32 includes an upwardly extending cylinder 36. When the material utilized to form the carrier 10 is poured into the form 32, the cylinder 36 operates to prevent the material from occupying the space taken by the cylinder 36 and to form the cavity 18 within the carrier 10. Also, as stated previously, while the preferred shape of the cavity 18 is cylindrical, the cylinder 36 in the form 32 can be altered to have any desired shape that results in the desired shape for the cavity 18.

Looking now at FIGS. 2 and 6, the length of the legs 28 at each end of the mold 22 are selected to have an overall length slightly larger than the length of the forms 32 extending down from the panel 24. In addition, the legs 28 have a width slightly less than the width of the opposed ends of each of the panel 24 and the peripheral rim 26. The reason for this particular preferred configuration for the legs 28 is such that legs 28 can be positioned on the panel 24 of an adjacent mold 22 within the rim 26 on the adjacent mold 22 and to enable the molds 22 to be positioned in a stacked configuration, as shown in FIG. 6. In this configuration, the only contact between the vertically adjacent molds is the legs 28, and the length of the legs 28 is sufficient to maintain the position of the forms 32 on the upper mold 22 above the central panel 24 of the lower mold 22.

In addition, as stated previously the carrier 10 formed by the mold 22 can have any desired size depending upon the particular individual who is to ingest the carrier 10 and the medication 21 contained therein. To facilitate the formation of carriers 10 have different sizes, the forms 32 and apertures 30 positioned on a panel 24 can be formed to have different sizes in order to form different size carriers 10 to be utilized by different types of individuals. Additionally, the number and size of the apertures 30 and forms 32 can vary as necessary to maximize the number of carriers 10 and the respective sizes of the carriers 10 to be formed by each mold 22. In order to further maximize the number of carriers 10 that can be formed by a given mold 22, the shape of the central panel 24 and accompanying peripheral rim 26 can be varied from the hexagonal shape shown in the preferred embodiments in FIGS. 2 and 6, to any desired shape. Also, depending upon the particular shape for the central panel 24, the number of legs 28 supporting the panel 24 can be varied in order to provide a stable base for the panel 24 and mold 22.

Referring now to FIG. 4, the carrier 10 can be formed of any suitable material that can be poured or otherwise flow into the form 32 through the aperture 30 in the central panel 24 and subsequently solidify or gel to form the carrier 10. While the preferred embodiment utilizes a gelatin material that is liquid at elevated temperatures but solidifies at room temperature, any other suitable material that is a liquid at elevated temperatures and a solid at room temperatures, and that is also ingestible may also be utilized.

Various alternatives are contemplated as being within the scope of the following claims particularly pointing out and distinctly claiming the subject matter regarded as the invention.

What is claimed is:

1. An ingestible carrier for a medication, the carrier comprising a body formed of an ingestible material and having a cavity formed therein with an open end and a closed end, wherein a cavity has a diameter that enables the cavity to frictionally engage a medication positioned therein.

2. The carrier of claim 1 wherein the open end of the cavity is disposed in a tapered side of the carrier.

3. The carrier of claim 2 wherein the carrier has a flat side opposite the tapered side.

4. The carrier of claim 3 wherein the carrier is bullet-shaped.

5. The carrier of claim 1 wherein the ingestible material is a liquid at elevated temperatures, and a solid at room temperature.

6. The carrier of claim 5 wherein the ingestible material is a gelatin.

7. The carrier of claim 1 wherein the cavity is generally cylindrical in shape.

8. A method for forming an ingestible carrier for a medication, the method comprising the steps of:
a) providing a mold including a central panel with at least one aperture and at least one mold form extending downwardly from the central panel around the at least one aperture, the at least one mold form having a cavity formed positioned therein;
b) pouring an ingestible material that is a liquid at elevated temperatures and a solid at room temperature in a liquid form through the at least one aperture into the at least one mold form around the cavity form; and
c) cooling the ingestible material into solid form.

9. The method of claim 8 further comprising the step of removing the carrier from the at least one mold form after allowing the ingestible material to change into a solid form.

10. The method of claim 8 wherein the step of pouring the ingestible material in liquid form comprises:
a) pouring the ingestible material onto the central panel; and
b) allowing the ingestible material to flow from the central panel into the at least one mold form through the at least one aperture.

11. A method for preparing an oral medication for ingestion, the method comprising the steps of:
a) providing a carrier formed of an ingestible material and including a cavity therein having an open end and a closed end; and
b) positioning a first medication within the cavity through the open end.

12. The method of claim 11 wherein the step of positioning the first medication within the cavity comprises frictionally engaging the first medication with a periphery of the cavity.

13. The method of claim 11 further comprising the step of positioning a second medication within the cavity after positioning the first medication within the cavity.

14. A mold for forming an ingestible carrier for a medication, the mold comprising:
a) a central panel including a number of apertures therein;
b) a number of mold forms connected to the central panel around the number of apertures; and
c) a number of cavity forms positioned within the number of mold forms and extending from the mold forms toward the number of apertures.

15. The mold of claim 14 wherein the central panel includes a number of first apertures and first mold forms, and a number of second apertures and second mold forms.

16. The mold of claim 15 wherein the first apertures and first mold forms and the second apertures and second mold forms are different sizes.

17. The mold of claim 16 wherein the cavity forms positioned within the first mold forms and the second mold forms are the same size.

18. The mold of claim 14 further comprising a peripheral rim disposed around the central panel.

19. The mold of claim 14 further comprising a pair of legs extending from the central panel to support the central panel.

20. The mold of claim 19 wherein the pair of legs on a first mold are engageable with the central panel of a second mold to position the first mold and the second mold in a stacked configuration.
